SEQUENCE LISTING (5 sequences)

SEQ ID NO: 1 - protein, 19 aa, Human
NRNGKETKSNYRVRVYQIP

SEQ ID NO: 2 - protein, 19 aa, Murine
NRRGKEVKSNYRVRVYQIP

SEQ ID NO: 3 - protein, 19 aa, Porcine
SRNGKETKSNYRVQVYQIP

SEQ ID NO: 4 - protein, 19 aa, Artificial
  Synthetic
NRRGKEVKSNYRVRVYQIC

SEQ ID NO: 5 - protein, 19 aa, Artificial
  Synthetic
NRRGKEVKSNYRVRVYQIC

(19) United States
(12) Patent Application Publication
(10) Pub. No.: US 2011/0311448 A1
(43) Pub. Date: Dec. 22, 2011

(54) ANTIBODY TO RAGE AND USES FOR IN VIVO IMAGING OR FOR TARGETING THERAPY

(76) Inventors: Ann Marie Schmidt, Franklin Lakes, NJ (US); Lynne Johnson, New York, NY (US); Barry Hudson, New York, NY (US); Yared Tekabe, Yonkers, NY (US)

(21) Appl. No.: 12/734,449

(22) PCT Filed: Oct. 31, 2008
(86) PCT No.: PCT/US08/12374

§ 371 Date: Oct. 12, 2010

Related U.S. Application Data
(60) Provisional application No. 61/001,598, filed on Nov. 2, 2007.

(51) Int. Cl. A61K 49/00; A61P 9/10; A61K 39/395; C07K 16/00; C07K 16/18

(52) U.S. Cl. 424/9.1; 530/387.9; 530/391.3; 530/391.7; 424/178.1

(57) ABSTRACT

This invention discloses an antibody which is raised to a peptide, the sequence of which is: N-R-N-G-K-E-T-K-S-N-Y-R-V-R-V-Y-Q-I-P, N-R-R-G-K-E-V-K-S-N-Y-R-V-R-V-Y-Q-I-P, S-R-N-G-K-E-T-K-S-N-Y-R-V-Q-V-Y-Q-I-P, N-R-R-G-K-E-V-K-S-N-Y-R-V-R-V-Y-Q-I-C, or Ac-N-R-R-G-K-E-V-K-S-N-Y-R-V-R-V-Y-Q-I-C-amide. This antibody can be labeled with an imageable marker or linked to an agent. This antibody can be a monoclonal antibody. The invention also includes a method for determining the location of receptor advanced glycation endproduct (RAGE) in a subject comprising administering the antibody labeled with an imageable marker and detecting the location of the labeled antibody in the subject thereby determining the location of RAGE in the subject. This invention further describes a method for treating a RAGE-related disorder in a subject comprising administering to the subject a therapeutically effective amount of the antibody which binds to the above-described epitope linked to an agent.

This application claims priority of U.S. Provisional Application No. 61/001,598, filed Nov. 2, 2007, the contents of which are hereby incorporated by reference.

Throughout this application, various publications are referenced by Arabic number in parenthesis. Full citations for these publications may be found listed numerically at the end of the specification immediately following the Experimental Procedures section and preceding the claims section. The disclosures of these publications in their entireties are hereby incorporated by reference into this application in order to more fully describe the state of the art as known to those skilled therein as of the date of the invention described and claimed herein.

### BACKGROUND OF INVENTION

Cardiovascular disease affects approximately 60 million people in the United States alone. Although myocardial perfusion imaging has proven prognostic usefulness, there are patients with <50% stenoses and no perfusion defects who have acute ischemic events including sudden death.

RAGE is a member of the immunoglobulin superfamily expressed at low levels in adult tissues in homeostasis, but highly upregulated at sites of vascular pathology. Ligand-triggered RAGE-dependent cellular activation augments inflammatory responses and enhances cellular migration and proliferation (2-4). It has been demonstrated that upregulation of RAGE and its inflammatory ligands is a consistent observation in human and animal models of diabetes and atherosclerosis (5,6).

Administration of RAGE antagonists to rats or mice, both with and without diabetes, attenuates vascular injury and greatly attenuates the initiation and acceleration of atherosclerosis (7,8). These findings support key roles for RAGE in atherosclerosis.

### SUMMARY OF INVENTION

A radionuclide approach to image RAGE activity is provided which can, inter alia, serve as a new noninvasive tool to access and treat atherosclerotic lesions. This invention also discloses an antibody raised to a peptide the sequence of which is: N-R-N-G-K-E-T-K-S-N-Y-R-V-R-V-Y-Q-I-P (SEQ ID NO:1), N-R-R-G-K-E-V-K-S-N-Y-R-V-R-V-Y-Q-I-P (SEQ ID NO:2), S-R-N-G-K-E-T-K-S-N-Y-R-V-Q-V-Y-Q-I-P (SEQ ID NO:3), N-R-R-G-K-E-V-K-S-N-Y-R-V-R-V-Y-Q-I-C (SEQ ID NO:4), or Ac-N-R-R-G-K-E-V-K-S-N-Y-R-V-R-V-Y-Q-I-C-amide (SEQ ID NO:5). This antibody can be labeled with an imageable marker or linked to an agent. The invention also includes a method for determining the location of receptor advanced glycation endproduct (RAGE) in a subject comprising administering the antibody labeled with an imageable marker and detecting the location of the labeled antibody in the subject thereby determining the location of RAGE in the subject. This invention further describes a method for treating a RAGE-related disorder in a subject comprising administering to the subject a therapeutically effective amount of the antibody which binds to the above-described epitope linked to an agent. This invention also discloses a pharmaceutical composition of the above-identified antibody linked to a therapeutic agent, and a pharmaceutically acceptable carrier. This invention discloses an imageable composition of the above-identified antibody linked to an imageable marker.

### BRIEF DESCRIPTION OF THE FIGURES

FIGS. 1A-1C. Anteroposterior planar gamma image of a 20 wk apoE^{-/-} mouse on Western-type diet with spontaneous atherosclerotic lesions 5 hours post intravenous injection of ^{99m}Tc-labeled anti-RAGE F(ab′)₂ (A). Uptake corresponding to the location of the atherosclerotic lesions is shown on the photograph (B). Comparison of radioactivity in the aortic lesions, the heart, and lungs from gamma scintillation counting (n=4) represented as % ID/g (C).

FIGS. 2A-2C. Anteroposterior planar gamma image of a 20 wk apoE^{-/-} mouse on Western-type diet with spontaneous atherosclerotic lesions 5 hours post intravenous injection of ^{99m}Tc-labeled nonspecific IgG F(ab′)₂ shows no tracer uptake in the thorax (A), although the in-situ dissection of the aortic arch shows extensive atherosclerotic plaque (B). Comparison of radioactivity in the aortic lesions, the heart, and lungs from gamma scintillation counting (n=2) represented as % ID/g (C).

FIGS. 3A-3C. Anteroposterior planar gamma image of a wild-type control C57BL/6 mouse 5 hours post intravenous injection of ^{99m}Tc-labeled anti-RAGE F(ab′)₂ (A), and gross examination of the aorta revealed no lesions (B). Comparison of radioactivity in the proximal aorta, the heart, and lungs from gamma scintillation counting (n=2) represented as % ID/g (C).

FIGS. 4A-4B Biodistribution of ^{99m}Tc-labeled anti-RAGE F(ab′)₂ (A), (n=4) and nonspecific IgG F(ab′)₂ (B), (n=2) 5 hours post intravenous administration of the radiotracer in non target organs of 20 wk apoE^{-/-} mice.

FIG. 5. Histological and immunohistochemical characterization of atherosclerotic lesions in mice. H&E and RAGE staining in representative sections of aorta from apoE^{-/-} mice receiving radiolabeled anti-RAGE antibody (top), apoE^{-/-} mice receiving radiolabeled nonspecific antibody (middle), and wild-type control C57BL/6 mice (bottom) receiving radiolabeled anti-RAGE antibody. Original magnification, ×100.

FIGS. 6A-6B. Epifluorescent micrographs of 5-μm-thick paraffin section of the aortic sinus from an apoE^{-/-} mouse injected with ^{99m}Tc and rhodamine-labeled anti-RAGE F(ab′)₂ shows co-localization of the fluorescence with RAGE (A). Immunohistochemical stained subjacent sections of the aortic sinus with anti-RAGE IgG shows specific staining in the lesions (B). Original magnification, ×100.

FIGS. 7A-7C. Representative sagittal and coronal SPECT images obtained from 24 week old diabetic apoE^{-/-} mouse (A), non-diabetic apoE^{-/-} mouse (B), and control apoE^{-/-}/RAGE^{-/-} mouse (C) 4 hours post i.v. injection of ^{99m}Tc-labeled anti-RAGE F(ab′)₂. Corresponding lesion severity is shown by H&E staining of proximal aortic section (bottom panel). Diabetic apoE^{-/-} mice showed intense tracer uptake in the thorax compared with the uptake in non-diabetic apoE^{-/-} mice. Control apoE^{-/-}/RAGE−/− mice showed no localization of the radiotracer at the target and histological examination of the aorta revealed minimal lesions.

FIG. 8. Bar graph shows uptake of radiotracer in the proximal aorta expressed as mean % ID/g±SD for diabetic apoE^{-/-} mice, non-diabetic apoE^{-/-} mice, and control apoE^{-/-}/RAGE^{-/-} mice.

FIG. 9. Biodistribution of radiolabeled anti-RAGE F(ab′)₂ in nontarget organs of diabetic (open bar) and non-diabetic mice (black bar) 5-6 h after i.v. injection of the radiotracer.

FIGS. 10A-10B. Immunohistochemical characterization of atherosclerotic lesions in representative tissue sections from apoE^{-/-} mice. Serial sections were stained for RAGE, SMCs (α-actin), and macrophages (Mac-3) (A). Staining of serial sections from the proximal aorta identified higher expressions of macrophages, RAGE, and smooth muscle cells in diabetic apoE^{-/-} mice (Top panel) compared with non-diabetic mice (Bottom panel). The specificity of anti-RAGE antibody was confirmed by lack of staining of aortic lesions in control apoE^{-/-}/RAGE^{-/-} mice (B). The chromogen stains brown. Original magnification ×400

FIGS. 11A-11B. Correlation of macrophage (A) and RAGE (B) positive cells with in vivo uptake (MBq). Regression analyses of in vivo aortic scans from diabetic (blue circle) and non-diabetic apoE^{-/-} (open circle) mice demonstrated association between macrophage and RAGE content with in vivo aortic scan.

### DETAILED DESCRIPTION OF THE INVENTION

The invention discloses, herein, An antibody raised to a peptide the sequence of which is: N-R-N-G-K-E-T-K-S-N-Y-R-V-R-V-Y-Q-I-P (SEQ ID NO:1), N-R-R-G-K-E-V-K-S-N-Y-R-V-R-V-Y-Q-I-F (SEQ ID NO:2), S-R-N-G-K-E-T-K-S-N-Y-R-V-Q-V-Y-Q-I-P (SEQ ID NO:3), N-R-R-G-K-E-V-K-S-N-Y-R-V-R-V-Y-Q-I-C (SEQ ID NO:4), or Ac-N-R-R-G-K-E-V-K-S-N-Y-R-V-R-V-Y-Q-I-C-amide (SEQ ID NO:5), or said antibody linked to a therapeutic agent, or said antibody labeled with an imageable marker. In one embodiment, the antibody is raised to a peptide the sequence of which is Ac-N-R-R-G-K-E-V-K-S-N-Y-R-V-R-V-Y-Q-I-C-amide (SEQ ID NO:5) or said antibody linked to a therapeutic agent, or said antibody labeled with an imageable marker. In one embodiment, the antibody is a monoclonal antibody. In another embodiment, the monoclonal antibody is produced by the hybridoma cell line 548D491.1 (Strategic BioSolutions, DE).

In another embodiment the imageable marker is technetium-99m. In another embodiment, the imageable marker is rhodamine.

In one embodiment the therapeutic agent is the V-domain of RAGE, soluble RAGE (sRAGE), an isolated peptide from RAGE capable of inhibiting the interaction between amyloid-beta peptide and RAGE, ligand-binding domain of sRAGE or ligand-binding domain of EN-RAGE, ribozyme, or an antisense nucleic acid.

Further disclosed herein is a method for determining the location of receptor for advanced glycation endproduct (RAGE) in a mammal comprising: (a) administering to the mammal a suitable amount of an antibody raised to a peptide the sequence of which is: N-R-N-G-K-E-T-K-S-N-Y-R-V-R-V-Y-Q-I-P (SEQ ID NO:1), N-R-R-G-K-E-V-K-S-N-Y-R-V-R-V-Y-Q-I-P (SEQ ID NO:2), S-R-N-G-K-E-T-K-S-N-Y-R-V-Q-V-Y-Q-I-P (SEQ ID NO:3), N-R-R-G-K-E-V-K-S-N-Y-R-V-R-V-Y-Q-I-C (SEQ ID NO:4), or Ac-N-R-R-G-K-E-V-K-S-N-Y-R-V-R-V-Y-Q-I-C-amide (SEQ ID NO:5,) which antibody is labeled with an imageable marker; and (b) after a period of time sufficient to permit binding of the antibody to RAGE, detecting the location of the labeled antibody in the mammal; thereby determining the location of RAGE in the mammal.

In one embodiment, the labeled antibody is raised to a peptide, the sequence of which is Ac-N-R-R-G-K-E-V-K-S-N-Y-R-V-R-V-Y-Q-I-C-amide (SEQ ID NO:5). In one embodiment, the mammal is a human. In one embodiment, the antibody is a monoclonal antibody. In another embodiment, the monoclonal antibody is produced by the hybridoma cell line 548D491.1 (Strategic BioSolutions, DE).

In another embodiment the imageable marker is technetium-99m. In another embodiment, the imageable marker is rhodamine.

This invention also discloses a method for treating a RAGE-related disorder in a mammal comprising administering to the mammal a therapeutically effective amount of raised to a peptide the sequence of which is: N-R-N-G-K-E-T-K-S-N-Y-R-V-R-V-Y-Q-I-P (SEQ ID NO:1), N-R-R-G-K-E-V-K-S-N-Y-R-V-R-V-Y-Q-I-P (SEQ ID NO:2), S-R-N-G-K-E-T-K-S-N-Y-R-V-Q-V-Y-Q-I-P (SEQ ID NO:3), N-R-R-G-K-E-V-K-S-N-Y-R-V-R-V-Y-Q-I-C (SEQ ID NO:4), or Ac-N-R-R-G-K-E-V-K-S-N-Y-R-V-R-V-Y-Q-I-C-amide (SEQ ID NO:5), linked to a therapeutic agent, thereby treating a RAGE-related disorder in the mammal. In one embodiment, the labeled antibody is raised to a peptide, the sequence of which is Ac-N-R-R-G-K-E-V-K-S-N-Y-R-V-R-V-Y-Q-I-C-amide (SEQ ID NO:5).

In one embodiment, the mammal is a human. In one embodiment, the antibody is a monoclonal antibody. In another embodiment, the monoclonal antibody is produced by the hybridoma cell line 548D491.1 (Strategic BioSolutions, DE).

In one embodiment the therapeutic agent is the V-domain of RAGE, soluble RAGE (sRAGE), an isolated peptide from RAGE capable of inhibiting the interaction between amyloid-beta peptide and RAGE, ligand-binding domain of sRAGE or ligand-binding domain of EN-RAGE, ribozyme, or an antisense nucleic acid.

This invention also discloses a pharmaceutical composition comprising an antibody raised to a peptide the sequence of which is: N-R-N-G-K-E-T-K-S-N-Y-R-V-R-V-Y-Q-I-P (SEQ ID NO:1), N-R-R-G-K-E-V-K-S-N-Y-R-V-R-V-Y-Q-I-P (SEQ ID NO:2), S-R-N-G-K-E-T-K-S-N-Y-R-V-Q-V-Y-Q-I-P (SEQ ID NO:3), N-R-R-G-K-E-V-K-S-N-Y-R-V-R-V-Y-Q-I-C (SEQ ID NO:4), or Ac-N-R-R-G-K-E-V-K-S-N-Y-R-V-R-V-Y-Q-I-C-amide (SEQ ID NO:5), linked to a therapeutic agent and a pharmaceutically acceptable carrier. In one embodiment, the antibody is raise to a peptide, the sequence of which is Ac-N-R-R-G-K-E-V-K-S-N-Y-R-V-R-V-Y-Q-I-C-amide (SEQ ID NO:5). In one embodiment, the antibody is a monoclonal antibody. In another embodiment, the monoclonal antibody is produced by the hybridoma cell line 548D491.1 (Strategic BioSolutions, DE).

This invention also discloses an imageable composition comprising an antibody raised to a peptide the sequence of which is: N-R-N-G-K-E-T-K-S-N-Y-R-V-R-V-Y-Q-I-P (SEQ ID NO:1), N-R-R-G-K-E-V-K-S-N-Y-R-V-R-V-Y-Q-I-P (SEQ ID NO:2), S-R-N-G-K-E-T-K-S-N-Y-R-V-Q-V-Y-Q-I-P (SEQ ID NO:3), N-R-R-G-K-E-V-K-S-N-Y-R-V-R-V-Y-Q-I-C (SEQ ID NO:4), or Ac-N-R-R-G-K-E-V-K-S-N-Y-R-V-R-V-Y-Q-I-C-amide (SEQ ID NO:5), linked to an imageable marker. In one embodiment, the antibody is raise to a peptide, the sequence of which is Ac-N-R-R-G-K-E-V-K-S-N-Y-R-V-R-V-Y-Q-I-C-amide (SEQ ID NO:5). In one embodiment, the antibody is a monoclonal antibody. In another embodiment, the monoclonal antibody is produced by the hybridoma cell line 548D491.1 (Strategic BioSolutions, DE). In another embodiment the imageable marker is technetium-99m. In another embodiment, the imageable marker is rhodamine.

This invention discloses use of an antibody which is raised to a peptide, the sequence of which is: N-R-N-G-K-E-T-K-S-N-Y-R-V-R-V-Y-Q-I-P (SEQ ID NO:1), N-R-R-G-K-E-V-K-S-N-Y-R-V-R-V-Y-Q-I-P (SEQ ID NO:2), S-R-N-G-K-E-T-K-S-N-Y-R-V-Q-V-Y-Q-I-P (SEQ ID NO:3), N-R-R-G-K-E-V-K-S-N-Y-R-V-R-V-Y-Q-I-C (SEQ ID NO:4), or Ac-N-R-R-G-K-E-V-K-S-N-Y-R-V-R-V-Y-Q-I-C-amide (SEQ ID NO:5), linked to a therapeutic agent for the manufacture of a medicament for treating a RAGE-related disorder. In one embodiment, the antibody is raise to a peptide, the sequence of which is Ac-N-R-R-G-K-E-V-K-S-N-Y-R-V-R-V-Y-Q-I-C-amide (SEQ ID NO:5). In one embodiment, the antibody is a monoclonal antibody. In another embodiment, the monoclonal antibody is produced by the hybridoma cell line 548D491.1 (Strategic BioSolutions, DE).

In one embodiment, the therapeutic agent is the V-domain of RAGE, soluble RAGE (sRAGE), an isolated peptide from RAGE capable of inhibiting the interaction between amyloid-beta peptide and RAGE, ligand-binding domain of sRAGE or ligand-binding domain of EN-RAGE, ribozyme, or an antisense nucleic acid.

This invention discloses an antibody which is raised to a peptide, the sequence of which is: N-R-N-G-K-E-T-K-S-N-Y-R-V-R-V-Y-Q-I-P (SEQ ID No:1), N-R-R-G-K-E-V-K-S-N-Y-R-V-R-V-Y-Q-I-P (SEQ ID NO:2), S-R-N-G-K-E-T-K-S-N-Y-R-V-Q-V-Y-Q-I-P (SEQ ID NO:3), N-R-R-G-K-E-V-K-S-N-Y-R-V-R-V-Y-Q-I-C (SEQ ID NO:4), or Ac-N-R-R-G-K-E-V-K-S-N-Y-R-V-R-V-Y-Q-I-C-amide (SEQ ID NO:5), linked to an imageable marker, for use in in vivo imaging of a RAGE-related disorder. In one embodiment, the antibody is raised to a peptide, the sequence of which is Ac-N-R-R-G-K-E-V-K-S-N-Y-R-V-R-V-Y-Q-I-C-amide (SEQ ID NO:5). In one embodiment, the antibody is a monoclonal antibody. In another embodiment, the monoclonal antibody is produced by the hybridoma cell line 548D491.1 (Strategic BioSolutions, DE). In another embodiment the imageable marker is technetium-99m. In another embodiment, the imageable marker is rhodamine.

### Terms

As used herein “RAGE” means a receptor for advanced glycation end products; “sRAGE” means a soluble form of a receptor for an advanced glycation end products, such as the extracellular two-thirds of the RAGE polypeptide, specifically the V and C domains.

As used herein, “antibody” means an immunoglobulin molecule comprising two heavy chains and two light chains and which recognizes an antigen. The immunoglobulin molecule may derive from any of the commonly known classes, including but not limited to IgA, secretory IgA, IgG and IgM. IgG subclasses are also well known to those in the art and include but are not limited to human IgG1, IgG2, IgG3 and IgG4. It includes, by way of example, both naturally occurring and non-naturally occurring antibodies. Specifically, “antibody” includes polyclonal and monoclonal antibodies, and monovalent and divalent fragments thereof. Furthermore, “antibody” includes chimeric antibodies, wholly synthetic antibodies, single chain antibodies, and fragments thereof. Optionally, an antibody can be labeled with a detectable marker. Detectable markers include, for example, radioactive or fluorescent markers. The antibody may be a human or nonhuman antibody. The nonhuman antibody may be humanized by recombinant methods to reduce its immunogenicity in man. Methods for humanizing antibodies are known to those skilled in the art.

As used herein, “imageable marker” means a medically acceptable composition that may be covalently or non-covalently linked to an antibody and which generates a signal detectable as a human perceivable visual signal, an electromagnetic signal, a radioactive signal or a signal detectable by magnetic resonance imaging, positron emission tomography or computerized axial tomography as is known in the art.

The imageable marker may be a radionuclide or a fluorophore. Examples of such markers may include, but are not limited to radionuclides such as indium-111, iodine-123, iodine 124, iodine-125, iodine 131, carbon-11, fluorine-18, copper-64 and technetium-99 and fluorophores such as rhodamine, fluorochromes (e.g., NIR fluorochromes such as Cy5™, Cy5.5™, Cy7™ or Licor NIR™, Alexa Fluor® 680, Alexa Fluor® 700, Alexa Fluor® 750, IRDye38™, IRDye78™, IRDye80™, indocyanine green, LaJolla Blue™, and Licor NIR™.

The labeling of the antibody or binding fragment can be accomplished by covalently or non-covalently linking the antibody to a moiety which generates an input for imaging techniques. Labeling may be performed by conventional techniques, including via chelating compounds, as described in, e.g., U.S. Pat. Nos. 4,741,900 and 4,986,979, each of which is incorporated by reference herein.

As used herein, “monoclonal antibody,” is used to describe antibody molecules whose primary sequences are essentially identical and which exhibit the same antigenic specificity. Monoclonal antibodies may be produced by hybridoma, recombinant, transgenic or other techniques known to one skilled in the art. Techniques to generate monoclonal antibodies can be found in Howard G C and Kaser M R, “Making and Using Antibodies” (2006) CRC Press, pages 73-92.

As used herein, “Fab” means a monovalent antigen binding fragment of an immunoglobulin that consists of one light chain and part of a heavy chain. It can be obtained by brief papain digestion or by recombinant methods.

As used herein, “F(ab′)2 fragment” means a bivalent antigen binding fragment of an immunoglobulin that consists of both light chains and part of both heavy chains. It can be obtained by brief pepsin digestion or recombinant methods.

As used herein, “epitope” means a portion of a molecule or molecules that forms a surface for binding antibodies or other compounds. The epitope may comprise contiguous or noncontiguous amino acids, carbohydrate or other nonpeptidyl moities or oligomer-specific surfaces.

“Administering” a compound can be effected or performed using any of the various methods and delivery systems known to those skilled in the art. The administering can be performed, for example, intravenously, orally, nasally, via the cerebrospinal fluid, via implant, transmucosally, transdermally, intramuscularly, intraocularly, topically and subcutaneously. The following delivery systems, which employ a number of routinely used pharmaceutically acceptable carriers, are only representative of the many embodiments envisioned for administering compositions according to the instant methods.

Injectable drug delivery systems include solutions, suspensions, gels, microspheres and polymeric injectables, and can comprise excipients such as solubility-altering compounds (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating compounds (e.g., starch polymers and cellulosic materials) and lubricating compounds (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending compounds (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking compounds, coating compounds, and chelating compounds (e.g., EDTA).

In the practice of the method, administration may comprise daily, weekly, monthly or hourly administration, the precise frequency being subject to various variables such as age and condition of the subject, amount to be administered, half-life of the compound in the subject, area of the subject to which administration is desired and the like.

As used herein, “RAGE-related diseases” shall mean diseases associated with an increased production of ligands for RAGE or with increased production of RAGE itself. These disorders may include, but are not limited to, many chronic inflammatory diseases, such as rheumatoid and psoriatic arthritis and intestinal bowel disease, cancers, diabetes and diabetic nephropathy, amyloidoses, atherosclerosis, sepsis, Alzheimers'Disease, senility, renal failure, neuronal cytotoxicity, Down's syndrome, dementia associated with head trauma, amyotrophic lateral sclerosis, multiple sclerosis or neuronal degeneration, an autoimmune disease, male impotence, wound healing, periodontal disease, neuopathy, retinopathy, nephropathy or neuronal degeneration.

As used herein, “therapeutic agent” shall mean any chemical entity, including, without limitation, a glycomer, a protein, an antibody, a lectin, a nucleic acid, a small molecule, and any combination thereof. Therapeutic agents used to treat RAGE-related diseases may include, but are not limited to, the V-domain of RAGE, soluble RAGE (sRAGE), an isolated peptide from RAGE capable of inhibiting the interaction between amyloid-beta peptide and RAGE, ligand-binding domain of sRAGE or ligand-binding domain of EN-RAGE, ribozyme, or an antisense nucleic acid.

“Therapeutically effective amount” of a compound means an amount of the compound sufficient to treat a subject afflicted with a disorder or a complication associated with a disorder. The therapeutically effective amount will vary with the subject being treated, the condition to be treated, the compound delivered and the route of delivery. A person of ordinary skill in the art can perform routine titration experiments to determine such an amount. Depending upon the compound delivered, the therapeutically effective amount of compound can be delivered continuously, such as by continuous pump, or at periodic intervals (for example, on one or more separate occasions). Desired time intervals of multiple amounts of a particular compound can be determined without undue experimentation by one skilled in the art.

“Amino acid residue” means an individual monomer unit of a polypeptide chain, which result from at least two amino acids combining to form a peptide bond.

“Amino acid” means an organic acid that contains both an amine group and a carboxyl group.

As used herein, the following standard abbreviations are used throughout the specification to indicate specific amino acids:

| | |
|---|---|
| A = ala = alanine | R = arg = arginine |
| N = asn = asparagine | D = asp = aspartic acid |
| C = cys = cysteine | Q = gln = glutamine |
| E = glu = glutamic acid | G = gly = glycine |
| H = his = histidine | I = ile = isoleucine |
| L = leu = leucine | K = lys = lysine |
| M = met = methionine | F = phe = phenylalanine |
| P = pro = proline | S = ser = serine |
| T = thr = threonine | W = trp = tryptophan |
| Y = tyr = tyrosine | V = val = valine |

“Peptide,” “polypeptide” and “protein” are used interchangeably herein to describe protein molecules that may comprise either partial or full-length sequences of amino acid residues.

“Treating” a disorder shall mean slowing, stopping or reversing the disorder's progression. In the preferred embodiment, treating a disorder means reversing the disorder's progression, ideally to the point of eliminating the disorder itself.

This invention provides the above compositions and a pharmaceutically acceptable carrier. Pharmaceutically acceptable carriers are well known to those skilled in the art. Such pharmaceutically acceptable carriers may include but are not limited to aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like.

### EXPERIMENTAL DETAILS

### First Series of Experiments

### Materials and Methods

### Development of Anti-RAGE Antibody

A novel antibody in rabbits was developed against the V-domain of RAGE designed to display immunoreactivity in mice, pigs and human. Based on Genbank sequences of human, murine and porcine RAGE, the following sequence alignment was determined and peptide identified below by SEQ ID NO:4 was prepared (9).

Human

| | |
|---|---|
| ----103-NRNGKETKSNYRVRVYQIP-121 | (SEQ ID NO: 1) |
| Murine | |
| ----102-NRRGKEVKSNYRVRVYQIP-120 | (SEQ ID NO: 2) |
| Porcine | |
| ----102-SRNGKETKSNYRVQVYQIP-120 | (SEQ ID NO: 3) |
| Peptide | |
| 1-NRRGKEVKSNYRVRVYQIC-19 | (SEQ ID NO: 4) |

This peptide was injected into rabbits and one rabbit displayed optimal titers of antibody; serum was retrieved, IgG prepared and then affinity-purified. Western blotting performed on lung extract from mouse and human revealed that this antibody recognized human, murine and porcine RAGE (10). Additionally, a monoclonal antibody which binds to the above-defined epitope has been produced.

### Preparation of F(ab′)₂ Fragments and Radiolabeling

Purified antibodies were subjected to digestion with immobilized pepsin beads using a kit from Pierce Chemical Co. (Rockford, Ill.). F(ab′)₂ fragments are superior to Fab because there are more antigen binding sites available, and faster blood pool and renal clearance compared to whole antibody. Direct coupling of anti-RAGE F(ab′)₂ antibodies to diethylenetriaminepentaacetic acid (DTPA) (Sigma Chemical Co.) for ^{99m}Tc labeling was performed as described (11). The immunoreactivity of DTPA modified antibody was tested by ELISA using soluble RAGE antigen-coated microtiter plates. Binding of the anti-RAGE F(ab′)₂ to the receptor was compared with that of unmodified anti-RAGE IgG using horseradish peroxidase (HRP)-conjugated secondary anti-rabbit IgG. The antibody concentration, which gave 50% of maximum binding with anti-RAGE F(ab′)₂ was 0.9 μg/ml, which is equivalent to 9×10⁻⁹ moles/L or apparent affinity of 0.11×10⁹ L/mole. The 50% of maximum binding concentration of unmodified anti-RAGE IgG was 0.8 μg/ml, which is equivalent to 8×10⁻⁹ moles/L or apparent affinity of 0.12×10⁹ L/mole.

For radiolabeling, an aliquot of modified anti-RAGE F(ab′)₂ (1-2 mg) was reacted with 5-fold molar excess of bicyclic anhydride of DTPA in 0.5 ml of dimethyl sulfoxide (DMSO) for 30 min at room temperature while stirring. The reaction mixture was dialyzed against excess (4 L) 0.1 mol/L NaHCO₃ in 0.1 mol/L NaCl, pH 7.6 at 4° C. overnight. An approximate 50-100 μg aliquot of DTPA modified anti-RAGE F(ab′)₂ was reacted with 1,296 MBq (30 mCi) of ^{99m}Tc—O⁻₄ in 50 μg of SnCl₂ in 100 μl of 0.1 N HCl that was flushed with N₂ for 20 min. After 30 min of incubation, the ^{99m}Tc-anti-RAGE F(ab′)₂ was separated from free ^{99m}Tc by Sephadex-G25 (10 ml) column (Pharmacia) equilibrated with PBS. Fractions (1.0 ml) were collected, and those fractions containing ^{99m}Tc-anti-RAGE F(ab′)₂ in the void volume were pooled. The mean specific activity was 53.1±18.9 μCi/μg, and the mean radiochemical purity was 94±4% by instant thin-layer chromatography. The mean injected ^{99m}Tc dose was 20.7±5.8 MBq.

Nonspecific control IgG was prepared from nonimmune rabbit serum, fragmented into F(ab′)₂, and coupled to DTPA for ^{99m}Tc labeling as described above.

### Preparation of Rhodamine-Labeled DTPA-Anti-RAGE F(ab′)₂

In order to localize the antibody uptake in vivo by histology, DTPA-labeled anti-RAGE F(ab′)₂ was conjugated to rhodamine isothiocyanate (Pierce Chemical Co.) and purified as previously reported. (12) The rhodamine-labeled DTPA-anti-RAGE F(ab′)₂ was radiolabeled as described above.

Blood Clearance of ^{99m}Tc-Labeled Anti-RAGE F(ab′)₂

Blood pool clearance study in mice was performed to determine the optimal time for imaging after injection of the ^{99m}Tc-labeled anti-RAGE F(ab′)₂. Two 20 wk old C57BL/6 mice were anesthetized with inhaled isoflurane (1.5% isoflurane at a flow of 0.5 L/min oxygen per mouse) and injected with 20.7 MBq (479 μCi) ^{99m}Tc-labeled anti-RAGE F(ab′)₂ antibody fragments. Blood samples (5 μl) were collected in capillary tubes via the tail vein at 5, 15, 20, and 30 min and 1, 3, 4, 5 and 6 h and radioactivity counted in a gamma counter (Wallac Wizard 1470, PerkinElmer, Waltham, Mass.).

### In-Vivo and Ex-Vivo Imaging

Male apoE^{-/-} mice (backcrossed >10 generations in the C57BL/6 background) were purchased from the Jackson Laboratories (Bar Harbor, Me.). At age 6 wk, 7 apoE^{-/-} mice were placed on Western-type diet (21%, w/w, fat [polyunsaturated/saturated ratio=0.07]) and 0.15%, w/w, cholesterol (Harlan Teklad, Madison, Wis.) for 14 wk. Corresponding wild-type male C57BL/6 mice on normal chow were used as controls. All animal studies were performed in accordance with the approval of the Institutional Animal Care and Use Committee of Columbia University.

At 20 wk of age, 5 apoE^{-/-} mice were anesthetized with inhaled isoflurane (1.5% isoflurane at a flow of 0.5 L/min oxygen per mouse) and injected with 20.7 MBq (479 μCi) ^{99m}Tc-labeled anti-RAGE F(ab′)₂ antibody fragments and the remaining 2 mice were injected with ^{99m}Tc-labeled control nonspecific IgG F(ab′)₂. Two control C57BL/6 mice were also injected with ^{99m}Tc-labeled anti-RAGE F(ab′)₂ and similarly imaged. Four hours later, the animals were re-anesthetized and serial whole body planar gamma images in the anteroposterior and lateral views were acquired each for 10 min on a high spatial resolution high sensitivity dedicated small animal camera with parallel-hole collimator (provided by Jefferson Lab, Newport News, Va.). The camera is based on a 5″ Hamamatsu position sensitive photomultiplier type R3292 with an active field-of-view of about 95 mm diameter. The scintillator sensor is 1.6 mm step 6 mm thick pixellated NaI (Tl) scintillator plate. The photo peak was set at 140 keV with a 15% energy window.

At the end of imaging, mice were euthanized by intraperitoneal injection of pentobarbitol (100 mg/kg). The aortic tree was dissected and photographed. Biodistribution studies were performed 5 h after injection of the ^{99m}Tc-labeled anti-RAGE F(ab′)₂ or nonspecific IgG F(ab′)₂. Tissues (aorta, heart, lung, liver, spleen, kidney, stomach, and small and large intestine) were dissected, washed with normal saline, weighed and counted in a gamma counter (Wallac Wizard 1470, PerkinElmer, Waltham, Mass.) for determination of the percent injected dose of radiotracer per gram (% ID/g) tissue.

Tracer uptake in the proximal aorta was quantified by using the region of interest (ROI) method in the mini gamma camera image. A region was drawn around the focal uptake and counts in the region were extracted using public domain ImageJ software (NIH, Bethesda, Md.). Percentage injected dose was calculated using corrections for isotope decay and camera efficiency and checked against comparing the counts in the aorta with counts in the total body which was in the field of view.

### Histopathology and Quantitative Morphometry

The heart and aorta were harvested by perfusion fixation for 10 min at physiologic pressure with formalin (10%). Tissues were fixed for 24 h in formalin (10%), followed by paraffin embedding. A 400 μm section of the proximal aorta from the aortic valve leaflets was excised. Serial 5-μm-thick sections of the aortic sinus were cut and every other section was collected. Sections were stained with hematoxylin-eosin (H&E) for morphology and for immunohistochemistry. Morphometric analyses of the arterial segments were performed using a Nikon microscope and image analysis system (Media Cybernetics Inc., Silver Spring, Md.). The amount of aortic lesion formation in each animal was measured as percent lesion area per total area of the aorta (13).

For cellular characterization, adjacent sections were deparaffinized in xylene, and treated with 0.3% hydrogen peroxide for 20 min to inactivate endogenous peroxidase. Tissue sections were then incubated in protein-free block (Dako, Carpinteria, Calif.) for 10 min to inhibit the nonspecific binding of primary antibody. Co-localization for RAGE was performed using polyclonal antibody to RAGE (50 μg/ml). Macrophages were identified using the marker Mac-3 (1:40; BD Pharmingen, San Diego, Calif.). Smooth muscle cells (SMCs) were localized using a primary antibody HHF-35 against α-actin (1:250; Sigma). Control immunostaining was performed using the respective nonspecific IgG. Detection was performed with HRP-conjugated goat anti-rabbit IgG (for RAGE) (Sigma), and mouse anti-rat IgG (for macrophages) (Serotec), and goat anti-mouse IgG (for SMC), followed by diaminobenzidine (DAB substrate kit for peroxidase, Vector Laboratories) and counterstaining with Gill's No. 3 hematoxylin solution.

The gamma imaging modality used was planar and not SPECT. The signal from the atherosclerotic lesion was strong and there was little lung activity. Photomicrographs of the aortic sinus and proximal aorta were taken with a digital camera mounted on a light microscope (Nikon, Tokyo, Japan). Pictures were digitalized and transferred to a personal computer for planimetry using Image Pro Plus software (Media Cybernetics). All images were analyzed at 100-fold magnification. Areas of positive staining for RAGE, SMCs, and macrophages (n=3 experimental apoE^{-/-} mice, 2 antibody control apoE^{-/-} mice, and 2 C57BL/6 mice) were measured in multiple plaques per animal and results were expressed as percent positive staining plaque area.

### Results

Blood Clearance of ^{99m}Tc-Labeled Anti-RAGE F(ab′)₂

Blood pool clearance curves showed a biexponential relationship. By 4 h after injection, blood pool cleared to below 20% of peak, thus indicating a sufficient reduction in background activity in order to allow visualization of target.

### In-Vivo Scans

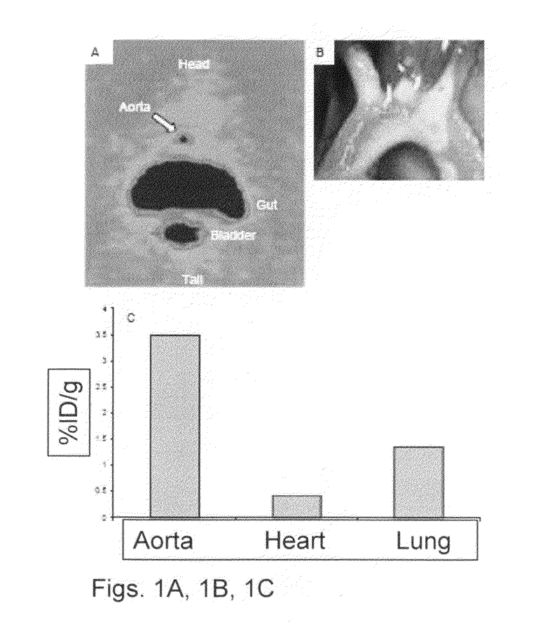

All five atherosclerotic apoE^{-/-} mice injected with ^{99m}Tc-labeled anti-RAGE F(ab′)₂ showed focal tracer uptake in the proximal aorta corresponding to the location of the atherosclerotic lesions seen at necropsy. An example from one experiment is shown in FIGS. 1A and 1B. The in-vivo scan findings were confirmed by well counting of the excised proximal aorta, heart and lungs (FIG. 1C). From ROIs drawn on the scans, the mean percent count in the experimental apoE^{-/-} mice was 0.991% (range, 0.36-1.54).

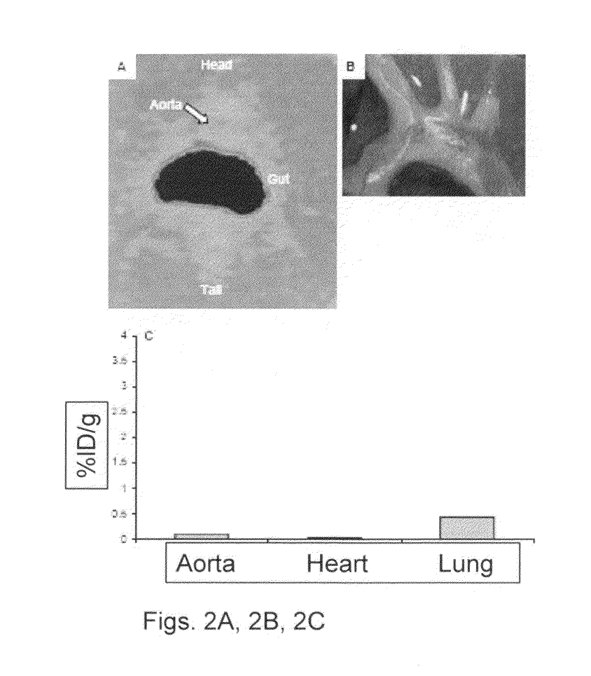

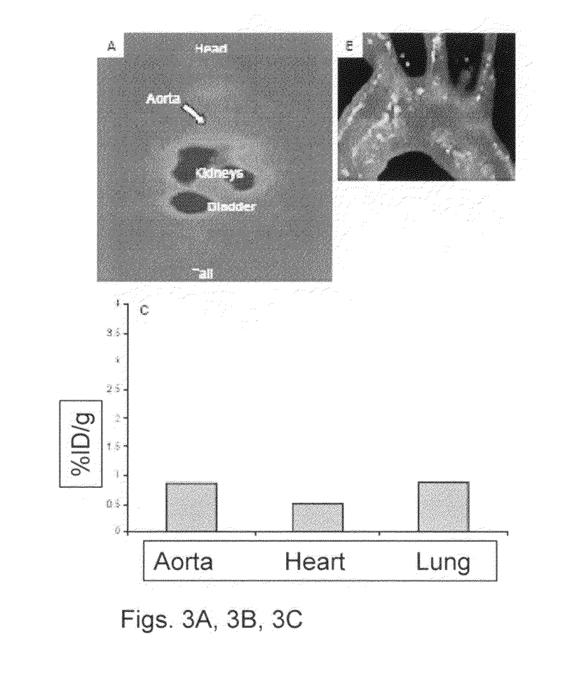

Atherosclerotic apoE^{-/-} mice injected with ^{99m}Tc-labeled nonspecific IgG F(ab′)₂ showed no tracer uptake in the thorax although the in-situ dissection of the aortic arch showed extensive atherosclerotic plaque (FIGS. 2A and 2B). These findings were confirmed by well counting of the excised tissue (FIG. 2C). Control C57BL/6 mice injected with ^{99m}Tc-labeled anti-RAGE F(ab′)₂ also showed no localization of the radiotracer at the target and gross examination of the aorta revealed no lesions (FIGS. 3A and 3B). Well counting confirmed the low signal (FIG. 3C). The mean percent count calculated from ROIs drawn from the in-vivo images were for the antibody control apoE^{-/-} mice 0.116% (average, 0.105, 0.128), and for the control C57BL/6 mice 0.367% (average, 0.29, 0.44).

### Biodistribution Studies

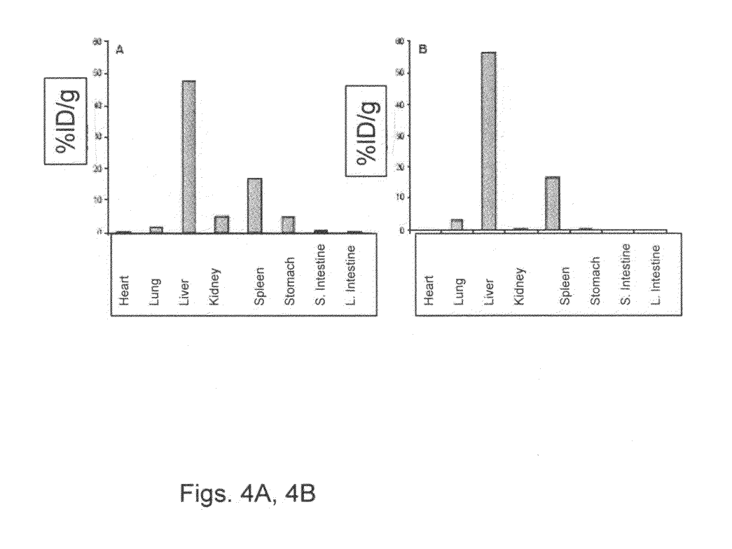

Biodistribution of radiolabeled anti-RAGE F(ab′)₂ (n=4) and nonspecific IgG F(ab′)₂ (n=2) in non-target organs performed by well counting of harvested tissue are shown in FIGS. 4A and 4B, respectively. In both groups, the mean percent injected dose per gram (% ID/g) activity in the liver was the greatest as was noted on the in-vivo scans.

### Quantitative Analysis of Atherosclerotic Lesions

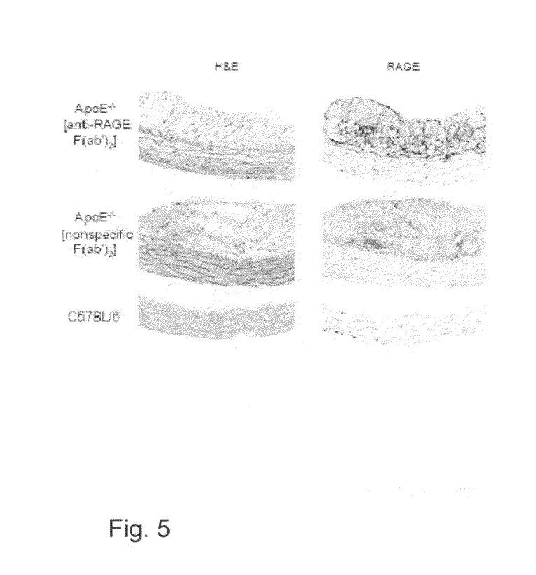

Histological sections through the proximal aorta in the apoE^{-/-} mice showed AHA class III lesions. The mean cross sectional area of the proximal aortic lesions as percent lesion area per total area of the aorta was 30.6% (range, 30.1-32.3%) (experimental apoE^{-/-} mice) versus 34.1% (average, 33.2, 35.1%) (antibody control apoE^{-/-} mice). The control C57BL/6 mice showed normal aortas without lesions. Immunohistochemical staining of the apoE^{-/-} aortas showed positive staining for RAGE localized to areas of macrophages and smooth muscle cells. The percentage of cells staining positive for RAGE was 13.5% (range, 11-15) in all apoE^{-/-} mice (FIG. 5).

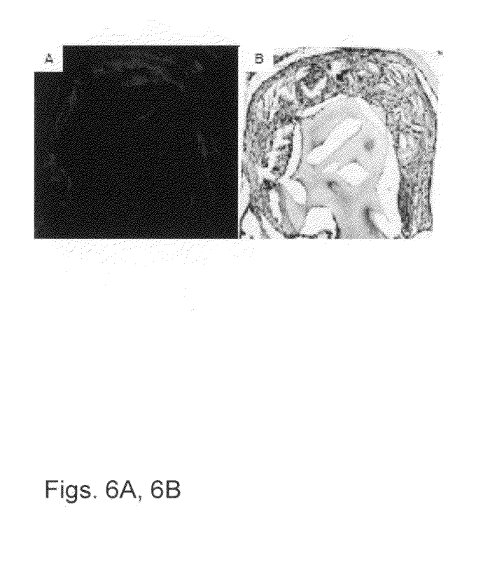

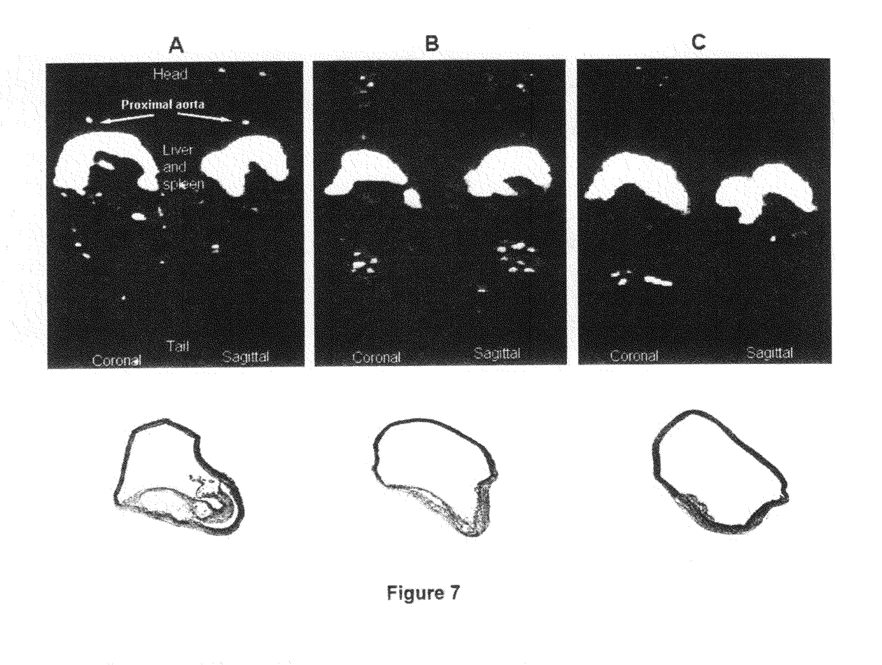

Dual labeling of the antibody with rhodamine showed co-localization of the fluorescence with RAGE. Immunohistochemical stained subjacent sections of the aortic sinus with anti-RAGE IgG shows specific staining in the lesion (FIG. 6).

### Discussion

In the present study, proof of concept that RAGE can be imaged in-vivo using a radiolabeled antibody was provided. Focal tracer uptake in the thorax with good target to background activity ratios corresponding to the location of the proximal aortic atheroma both by gross dissection and by biodistribution was visible by 4-5 h after administration of the radiolabeled antibody.

Advances in molecular biology over the past 10 years have identified potential sites in atherosclerotic plaque that can be targeted with probes that produce signals which may be detected using external imaging. Experimental and clinical studies have reported the feasibility of detecting signals from atherosclerotic plaque using nuclear medicine technology (1). Nuclear medicine uses probes in nanomolar concentrations that have no biological effects. Targets below the resolution of imaging devices can be detected as beacons if there are abundant binding sites and low background, or with tracers specially designed to amplify the signal.

Advanced Glycation End Products (AGEs) are formed by the nonenzymatic linkage of glucose to proteins and their formation is a direct consequence of prolonged levels of hyperglycemia in diabetes (14-17). In 1992 endothelial cell surface-associated proteins that mediate the interaction of AGEs with endothelium were first described (10, 18). Studies demonstrated AGE binding activity in bovine endothelial cells and lung extracts (19). NH₂-terminal sequence analysis indicated that one of the cell surface AGE binding site comprises an integral membrane protein, receptor for AGE (RAGE) (2). Binding of AGEs to receptors induces multiple signaling pathways involved in plaque initiation and progression. These receptors also bind non-AGE-related pro-inflammatory markers S100/calgranulins, amphoterin (also known as High Mobility Group Box-1), and amyloid-β peptide and β-sheet fibrils (3, 20). Because of this latter broader function in inflammatory mechanisms, these receptors are implicated in atherosclerosis in non-diabetic animals as well.

In previous studies, a soluble form of RAGE (sRAGE) that includes the extracellular ligand-binding domain was developed and tested, and showed that administration of this agent to diabetic and non-diabetic atherosclerotic mice reduced AGE-RAGE interaction and suppressed or stabilized atherosclerosis development (7,8).

RAGE is highly conserved across species and is widely distributed in vascular and lung tissue from non-diabetic animals with close homology to man. Immunostaining of bovine tissues demonstrated RAGE in the vasculature, endothelium, and smooth muscle cells and in mononuclear cells in the tissues (19). RAGE antigen and mRNA were found in cultured endothelium, vascular smooth muscle, and monocyte-derived macrophages. RAGE antigen was also visualized in bovine cardiac myocytes, neonatal rat cardiac myocytes and in neural tissue. Several reports using human material have reported RAGE expression in atherosclerotic plaques from both diabetic and non-diabetic patients. Although there were greater numbers of inflammatory cells (macrophages, T lymphocytes) and larger necrotic cores in diabetic lesions compared to non-diabetic lesions, RAGE expression correlated with inflammation and core size in all human samples (5, 21).

### REFERENCES

1. Davies J R, Rudd J H, Weissberg P L. Molecular and metabolic imaging of atherosclerosis. J Nucl Med. 2004; 45:1898-1907.
2. Kislinger T, Fu C, Huber B, Qu W, Taguchi A, Yan S D, Hofmann M, Yan S F, Pischetsrieder M, Stern D, Schmidt A M. N^{ε}-(carboxymethyl) lysine adducts of proteins are ligands for receptor for advanced glycation endproducts that activate cell signaling pathways and modulate gene expression. J Biol Chem. 1999; 274:31740-31749.
3. Hofmann M A, Drury S, Fu C, Qu W, Taguchi A, Lu Y, Avila C, Kambham N, Bierhaus A, Nawroth P, Neurath M F, Slattery T, Beach D, McClary J, Nagashima M, Morser J, Stern D, Schmidt A M. RAGE mediates a novel proinflammatory axis: a central cell surface receptor for S100/calgranulin polypeptides. Cell. 1999; 97:889-901.
4. Schmidt A M, Yan S D, Brett J, Mora R, Nowygrod R, Stern D. Regulation of human mononuclear phagocyte migration by cell surface binding proteins for AGE. J. Clin. Invest. 1993; 91:2155-2168.
5. Cipollone F, Iezzi A, Fazia M, Zucchelli M, Pini B, Cuccurullo C, De Cesare D, De Blasis G, Muraro R, Bei R, Chiarelli F, Schmidt A M, Cuccurullo R, Mezzetti A. The receptor RAGE as a progression factor amplifying arachidonate-dependent inflammatory and proteolytic response in human atherosclerotic plaques—Role of glycemic control. Circulation. 2003; 108:1070-1077.
6. Schmidt A. M, Du Yan S, Wautier J L, Stern D. Activation of receptor for advanced glycation endproducts: a mechanism for chronic vascular dysfunction in diabetic vasculopathy and atherosclerosis. Circ Res. 1999; 84:489-497.
7. Bucciarelli L G, Wendt T, Qu W, Lu Y, Lalla E, Rong L L, Goova M T, Moser B, Kislinger T, Lee D C, Kashyap Y, Stern D M, Schmidt A M. RAGE blockade stabilizes established atherosclerosis in diabetic apolipoprotein E-null mice. Circulation. 2002; 106:2827-2835.
8. Park L, Raman K G, Lee K J, Lu Y, Ferran L J, Chow W S, Stern D, Schmidt A M. Suppression of accelerated diabetic atherosclerosis by the soluble receptor for advanced glycation endproducts. Nat. Med. 1998; 4:1025-1031.
9. Neeper M, Schmidt A M, Brett J, Yan S D, Wang F, Pan Y C, Elliston K, Stern D, Shaw A. Cloning and expression of RAGE: a cell surface receptor for advanced glycosylation end products of proteins. J Biol Chem. 1992; 267:14998-15004.
10. Schmidt A M, Vianna M, Gerlach M, Brett J, Ryan J, Kao J, Esposito C, Hegarty H, Hurley W, Clauss M, Wang F, Pan Y C, Tsang T, Stern D. Isolation and characterization of binding proteins for advanced glycosylation end products from bovine lung which are present on the endothelial cell surface. J Biol Chem. 1992; 267:14987-14997.
11. Hnatowich D L, Layne W W, Childs R L, Lateinge D, Davis M A, Griffin T W, Doherty P W. Radioactive labeling of antibody: a simple and efficient method. Science. 1983; 220:613-615.
12. Khaw B A, Tekabe Y, Johnson L L. Imaging experimental atherosclerotic lesions in apoE knockout mice: enhanced targeting with Z2D3-anti-DTPA bispecific antibody and 99mTc-labeled negatively charged polymers. J Nucl Med. 2006; 47:868-877.
13. Daugherty A, Whitman S C. “Quantification of atherosclerosis in mice.” Methods in Molecular Biology, ed M. H. Hofker and J van Duersen. 2003; 209:293-307.
14. Basta G, Schmidt A M, De Caterina R. Advanced glycation endproducts and vascular inflammation: implications for accelerated atherosclerosis in diabetes. Cardiovasc Research. 2004; 63:582-592.
15. Falcone C, Emanuele E, D'Angelo A, Buzzi M P, Belvito C, Cuccia M, Geroldi D. Plasma levels of soluble receptor for advanced glycation end products and coronary artery disease in nondiabetic men. Arterioscler Thromb Vasc Biol. 2005; 25:1032-1037.
16. Hudson B I, Harja, E, Moser B, Schmidt A M. Soluble levels of Receptor for Advanced Glycation Endproducts (sRAGE) and coronary disease—The next C-reactive protein? Arterioscler Thromb Vasc Biol. 2005; 25:879-882.
17. Jandeleit-Dahm K A, Lassila M., Allen T J. Advanced glycation endproducts in diabetes-associated atherosclerosis and renal disease. Ann N.Y. Acad. Sci. 2005; 1043:759-766.
18. Schmidt A M, Hasu M, Popov D, Zhang J H, Chen J, Yan S D, Brett J, Cao R, Kuwabara K, Costache G, Simionescu N, Stern D. The Receptor for Advanced Glycation Endproducts (RAGEs) has a central role in vessel wall interactions and gene activation in response to circulating AGE proteins. Proc. Natl. Acad. Sci. USA 1994; 91:8807-8811.
19. Brett J, Schmidt A M, Yan S D, Zou Y S, Weidman E, Pinsky D, Nowygrod R, Neeper M, Przysiecki C, Shaw A, Migheli A, Stern D. Survey of the distribution of a newly characterized receptor for advanced glycation endproducts in tissues. Am J Pathol. 1993; 143:1699-1722.
20. Arumugam T, Simeone D M, Schmidt A M, Logsdon C D. S100P stimulates cell proliferation and survival via receptor for advanced glycation endproducts (RAGE). J Bio Chem. 2004; 279:5059-5065.
21. Burke A P, Kolodgie F D, Zieske A, Fowler D R, Weber D K, Varghese P J, Farb A, Virmani R. Morphologic findings of coronary atherosclerotic plaques in diabetes. Arterioscler Thromb Biol. 2004; 24:1266-1271.

### Second Series of Experiments

### Introduction

Receptor for Advanced Glycation Endproducts (RAGE) binds AGEs and other inflammatory ligands and is expressed in atherosclerotic plaques in diabetic and non-diabetic subjects. The higher expression in diabetes mellitus (DM) corresponds with accelerated course of the disease. This study was designed to test the hypothesis that the level of RAGE expression in atherosclerosis can be detected by quantitative in-vivo SPECT imaging and that counts in the target will correlate with the strength of the biologic signal.

Diabetes is becoming epidemic in the US and as a coronary artery disease risk factor is considered to be a coronary artery disease equivalent. Atherosclerosis in diabetics takes an accelerated course. Assessing total plaque burden is important to tailor individual patient therapy. Advances in molecular biology over the past 10 years have identified potential sites in atherosclerotic plaque that can be targeted with probes that produce signals that can be detected using external imaging. Experimental and clinical studies have reported the feasibility of detecting signals from atherosclerotic plaque using nuclear medicine technology. Nuclear medicine uses probes in nanomolar concentrations that have no biological effects. Targets below the resolution of imaging devices can be detected as beacons if there are abundant binding sites and low background.

RAGE expression plays a key role in initiation and acceleration of atherosclerosis in both diabetics and nondiabetics. RAGE is a member of the immunoglobulin superfamily expressed at low levels in adult tissues in homeostasis, but highly expressed at sites of vascular pathology. (1-3) Expression of RAGE and its inflammatory ligands is a consistent observation in human and animal models of diabetes and atherosclerosis. (4,5) Binding of AGEs to receptors induces multiple signaling pathways involved in plaque progression. These receptors also bind non-AGE-related pro-inflammatory markers S100/calgranulins, amphterins, EN-RAGE. (6,7) Because of this latter broader function these receptors are implicated in progression of atherosclerosis in non-diabetics. Administration of RAGE antagonists to rats or mice, both with and without diabetes, attenuates vascular injury and greatly attenuates the initiation and acceleration of atherosclerosis. (8,9) These findings support key roles for RAGE in diabetic atherosclerosis.

We have previously shown the ability to visualize uptake of an F(ab′)₂ fragment of a ^{99m}Tc-labeled polyclonal antibody in atherosclerotic plaque in the aortic root of 20 wk non-diabetic apoE^{-/-} mice using planar imaging. (10) The generation of monoclonal antibodies is now a standard and increasingly routine procedure. Since the hybridoma cell lines are immortal, there is an unlimited source of the monoclonal antibody. Monoclonal antibodies show reduced nonspecific binding and less nonspecific background activity compared to polyclonal antibodies and therefore we developed a monoclonal antibody directed against the same peptide sequence on the extracellular receptor domain of RAGE. The hypothesis of the present study was that in age matched apoE^{-/-} mice uptake of radiolabeled antibody fragment of the monoclonal anti-RAGE antibody would be greater in diabetic compared to non-diabetic mice and would be a marker of the accelerated course of atherosclerosis in these diabetic animals.

A monoclonal murine antibody was developed against the V-domain of RAGE, fragmented into F(ab′)₂ and labeled with ^{99m}Tc and dose of 15.14±1.23 MBq injected into 31 24 wk old apolipoprotein E null (apoE^{-/-}) mice 9 with streptozotocin-induced DM and 7 control apoE^{-/-}/RAGE^{-/-} double knock-out. Four hours later (blood pool clearance) mice were imaged on HiSPECT scanner (Bioscan), sacrificed, the proximal aorta removed, counted, and sectioned. Uptake in the thorax corresponding to the proximal aorta was quantified using Interview XP (Mediso) software. Lesion size and % cells staining positive for RAGE were quantified from tissue sections using immunohistomorphometry. Lesion morphology was AHA class II-III and lesion size was 20.7±9.5 (non-diabetic) and 37.1±16.1 (diabetic) (P=0.04). RAGE uptake (mean percent injected dose) in diabetic apoE^{-/-} mice (0.31±0.19) was significantly higher than the non-diabetic apoE^{-/-} (0.062±0.01; P=0.003) or control apoE^{-/-}/RAGE^{-/-} (0.031±0.006; P=0.002). Values for mean uptake (MBq) from scans correlated well with histology. When % RAGE positive cells and % macrophages were plotted against in vivo uptake (MBq) from scans and the correlations were both highly significant: R²=0.88 for both with P<0.0001 for both.

In this study ^{99m}Tc-labeled anti-RAGE F(ab′)₂ SPECT imaging successfully identified early accelerated disease in DM for age matched apoE^{-/-} mice and quantified RAGE expression over a range of lesion severities.

### Methods

### Experimental Model

Male apoE^{-/-} mice (backcrossed >10 generations in the C57BL/6 background) were purchased from the Jackson Laboratories (Bar Harbor, Me.). The RAGE^{-/-}/apoE^{-/-} mice were generated by backcrossing RAGE^{-/-} mice on the C57BL/6 background into apoE^{-/-} mice on the same background for 10 generations. (11) At age 6 wk, 9 apoE^{-/-} mice were made diabetic via 5 daily i.p. injection of streptozotocin (STZ, Sigma, St. Louis, Mo.) 50 mg/kg in citrate buffer (0.05 mol/L; pH 4.5) per day, resulting in insulin deficiency. (12) Control animals received citrate buffer only. All animals were studied at age 24 wks. All animal studies were performed in accordance with the approval of the Institutional Animal Care and Use Committee of Columbia University.

### Development of Monoclonal Anti-RAGE Antibody

We developed a novel antibody in rabbits against the V-domain of RAGE designed to display immunoreactivity in mice, pigs and human. Based on Genbank sequences of human, murine and porcine RAGE, the following sequence alignment was determined and peptide identified below by SEQ ID NO:4 was prepared. (13)

Human

| | |
|---|---|
| ----103-NRNGKETKSNYRVRVYQIP-121 | (SEQ ID NO: 1) |
| Murine | |
| ----102-NRRGKEVKSNYRVRVYQIP-120 | (SEQ ID NO: 2) |
| Peptide | |
| 1-NRRGKEVKSNYRVRVYQIC-19 | (SEQ ID NO: 4) |

Twenty mg of the peptide Ac-NRRGKEVKSNYRVRVYQIC-amide (SEQ ID NO:5) was produced by Quality Controlled Biochemicals (Hopkinton, Mass.). For hybridoma development, the peptide was conjugated to carrier molecule keyhole limpet hemocyanin (KLH) and 15 Balb C mice were immunized with the conjugated peptide with 3-6 injections over a 6 to 10 week period. (Strategic BioSolutions, Newark, Del.). Test bleeds were obtained after the fourth and subsequent immunizations to evaluate polyclonal antisera binding to RAGE antigen by ELISA screening. Fusion of the spleen cells to myeloma cells was successful to prepare the hybridoma cell lines and hybridoma culture supernatants were evaluated to identify positive hydbridomas. The hybridomas were sub-cloned and the best sub-clones showing the best supernatant binding to RAGE antigen as determined by ELISA were selected. Monoclonal antibodies were produced in vitro from hybridoma cell line 548D491.1 (produced by Strategic BioSolutions, DE) and purified by Protein A and low endotoxin units (less than 3 EU/mg of purified antibody). The purity of the monoclonal antibody (>95%) was determined by HPLC. The isoelectric point (6.4-6.9 μl range) was determined by isoelectric focusing. IgG isotype (IgG2a Kappa) was determined, using Isostrip™. Techniques to generate monoclonal antibodies can be found in Howard G C and Kaser M R, “Making and Using Antibodies” (2006) CRC Press, pages 73-92, the contents of which is hereby incorporated by reference.

### Preparation of F(ab′)₂ Fragments and Radiolabeling

F(ab′)₂ fragments of the purified antibody were prepared as previously described (10). These fragments have more antigen binding sites available than Fab, and faster blood pool and renal clearance compared to whole antibody. Direct coupling of anti-RAGE F(ab′)₂ antibodies to diethylenetriaminepentaacetic acid (DTPA) (Sigma Chemical Co.) for ^{99m}Tc labeling was performed as previously described. (10,14) The immunoreactivity of DTPA modified antibody was tested by ELISA using soluble RAGE antigen-coated microtiter plates. Binding of the anti-RAGE F(ab′)₂ to the receptor was compared with that of unmodified anti-RAGE IgG using horseradish peroxidase (HRP)-conjugated secondary anti-rabbit IgG. The antibody concentration, which gave 50% of maximum binding with anti-RAGE F(ab′)₂ was 0.2 μg/ml, which is equivalent to 2×10⁻⁹ moles/L or apparent affinity of 0.5×10⁹ L/mole. The 50% of maximum binding concentration of unmodified anti-RAGE IgG was 0.4 μg/ml, which is equivalent to 2×10⁻⁹ moles/L or apparent affinity of 0.5×10⁹ L/mole.

Radiolabeling anti-RAGE F(ab′)₂ with ^{99m}Tc was performed as described previously (10). Briefly, an aliquot of modified anti-RAGE F(ab′)₂ (1-2 mg) was reacted with 5-fold molar excess of bicyclic anhydride of DTPA in 0.5 ml of dimethyl sulfoxide (DMSO) for 30 min at room temperature while stirring. The reaction mixture was dialyzed against excess (4 L) 0.1 mol/L NaHCO₃ in 0.1 mol/L NaCl, pH 7.6 at 4° C. overnight. An approximate 50 μg aliquot of DTPA modified anti-RAGE F(ab′)₂ was reacted with 50-60 mCi of ^{99m}TcO₄⁻ in 50 μg of SnCl₂ in 100 μl of 0.1 N HCl that was flushed with N₂ for 20 min. After 30 min of incubation, the ^{99m}Tc-anti-RAGE F(ab′)₂ was separated from free ^{99m}Tc by Sephadex-G25 (10 ml) column (Pharmacia) equilibrated with PBS. Fractions (1.0 ml) were collected, and those fractions containing ^{99m}Tc-anti-RAGE F(ab′)₂ in the void volume were pooled. The mean specific activity was 178±18.6 mCi/μg of protein, and the mean radiopurity was 98±0.54% by instant thin-layer chromatography. The mean injected ^{99m}Tc dose was 15.14±1.23 MBq.

Blood Clearance of ^{99m}Tc-Labeled Anti-RAGE F(ab′)₂

Blood pool clearance study in mice was performed to determine the optimal time for imaging after injection of the ^{99m}Tc-labeled monoclonal anti-RAGE F(ab′)₂. Two 20 wk old C57BL/6 mice were anesthetized with inhaled isoflurane (1.5% isoflurane at a flow of 0.5 L/min oxygen per mouse) and injected with ⁹⁹Tc-labeled anti-RAGE F(ab′)₂ antibody fragments. Blood samples (2 μl) were collected in capillary tubes via the tail vein at 2, 10, 30, 60, 120, 180, 240, 360, 600, and 1440 min and radioactivity counted in a gamma counter (Wallac Wizard 1470, PerkinElmer, Waltham, Mass.).

### In Vivo High-Resolution SPECT

Whole-body multi-pinhole SPECT images (HiSPECT, Bioscan) were acquired with specially designed pyramidal collimators with 1.0-mm pinhole tungsten apertures. A triple-detector gamma camera (Prism 3000XP) was used to acquire photopeak for ^{99m}Tc imaging (140 KeV, 10% energy window) with the following parameters: step and shoot rotation, image acquisition time of 90 seconds per stop, 30° step in 360° rotation, and 15.3 cm radius of rotation. Images were acquired with 256×256 matrix and a reconstructed voxel size of 0.125 mm³. Region-of-interest (ROI) analysis was performed with customized software (Interview XP; Medisco).

### Image Analysis and Ex Vivo Counting

At the completion of imaging, the animals were euthanized by i.p. injection of pentobarbitol (100 mg/kg). The aortic tree was dissected and photographed. Biodistribution studies were performed 5-6 h after injection of the radiotracer. Tissues (aorta, heart, lung, liver, spleen, kidney, stomach, and small and large intestine) were dissected, washed with normal saline, weighed and counted in a gamma counter (Wallac Wizard 1470, PerkinElmer, Waltham, Mass.) for determination of the percent injected dose of radiotracer per gram (% ID/g) tissue.

### Histopathology and Quantitative Morphometry

The proximal aorta was harvested by perfusion fixation for 10 min with 10% neutral buffered formalin. Tissues were fixed for 24 h in 10% formalin, followed by paraffin embedding. A 400 μm section of the proximal aorta from the aortic valve leaflets was excised. Serial 5-μm-thick sections were cut and stained with hematoxylin-eosin (H&E) for morphology and for immunohistochemistry. Morphometric analyses of the arterial segments were performed using a Nikon microscope and image analysis system (Media Cybernetics Inc., Silver Spring, Md.). The amount of aortic lesion formation in each animal was measured as percent lesion area per total area of the aorta. (15)

For immunohistochemical staining, sections were deparaffinized in xylene, and endogenous peroxidase activity was blocked using 0.3% hydrogen peroxide. Tissue sections were then incubated in protein-free block (Dako, Carpinteria, Calif.) for 10 min to inhibit the nonspecific binding of primary antibody. Staining for RAGE was performed using antibody against RAGE (50 μg/ml). Macrophages were identified using the marker Mac-3 (1:20; BD Pharmingen, San Diego, Calif.). Smooth muscle cells (SMCs) were identified using a primary antibody HHF-35 against α-actin (1:250; Sigma). Control immunostaining was performed using the respective nonspecific IgG. Detection was performed with HRP-conjugated goat anti-rabbit IgG (for RAGE) (Sigma), mouse anti-rat IgG (for macrophages) (Serotec), and goat anti-mouse IgG (for SMC). Color was developed with 3′,3′-diaminobenzidine (DAB substrate kit, Vector Laboratories) and counterstaining with Gill's hematoxylin solution.

### Statistical Analysis

All data are presented as mean±standard deviation. Statistical comparison between groups was made by use of either paired or unpaired Student t test. Simple linear regression with the least-squares method was used to determine the relationship between histological findings with in vivo scan. Differences between groups were considered significant at a value of P<0.05.

### Results

Blood Clearance of ^{99m}Tc-Anti-RAGE F(ab′),

Blood pool clearance showed a bi-exponential curve. The t_{1/2} for the first component was 20 min and for the second component 7 h.

### In-Vivo Scans

All diabetic apoE^{-/-} mice injected with ^{99m}Tc-labeled anti-RAGE F(ab′)₂ showed focal tracer uptake in the thorax corresponding to the location of the proximal aortic atherosclerotic lesion. An example from one experiment is shown in FIG. 7A. Non-diabetic apoE^{-/-} mice also showed tracer uptake in the thorax corresponding to the proximal aorta but the uptake was less intense (FIG. 7B). Control apoE^{-/-}/RAGE^{-/-} mice showed no uptake of the radiotracer in the aortic region of the thorax and histological examination of the aorta revealed minimal lesions (FIG. 7C). From ROIs drawn on the scans around the focal uptake in the thorax, mean radiotracer uptake (as MBq) in the diabetic apoE^{-/-} group (1.60±0.61) was significantly greater than the uptake in corresponding locations in the non-diabetic apoE^{-/-} mice (0.48±0.26; P=0.001) or in control apoE^{-/-}/RAGE^{-/-} mice (0.31±0.07; P=0.0007 vs. apoE^{-/-} mice).

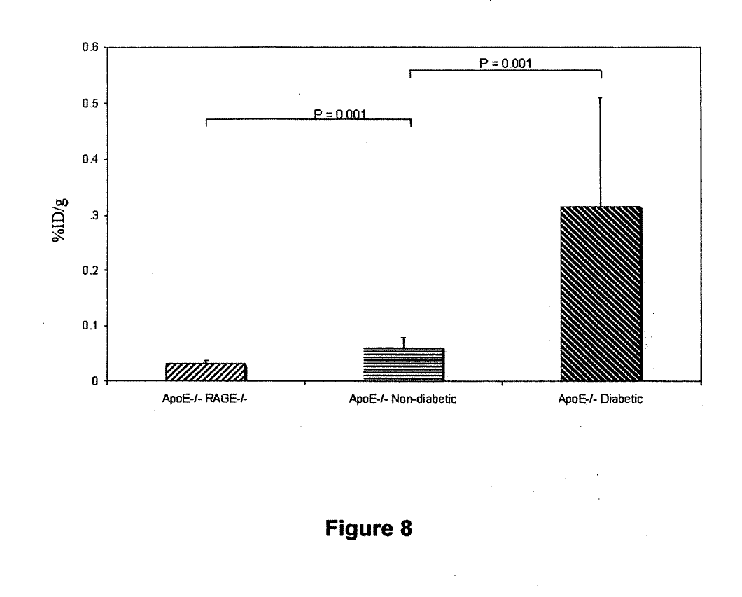

The radiotracer uptake (mean % ID/g) in the proximal aorta was confirmed by ex vivo gamma counting of aortic tissues. RAGE uptake in aortic segments in diabetic apoE^{-/-} mice (0.31±0.19) was significantly higher than the uptake in non-diabetic apoE^{-/-} (0.062±0.01; P=0.003), or in control apoE^{-/-}/RAGE^{-/-} (0.031±0.006; P=0.002) (FIG. 8). The radiotracer uptake in the proximal aorta in non-diabetic apoE^{-/-} mice was also significantly greater than the uptake in control apoE^{-/-}/RAGE^{-/-} mice (P=0.001).

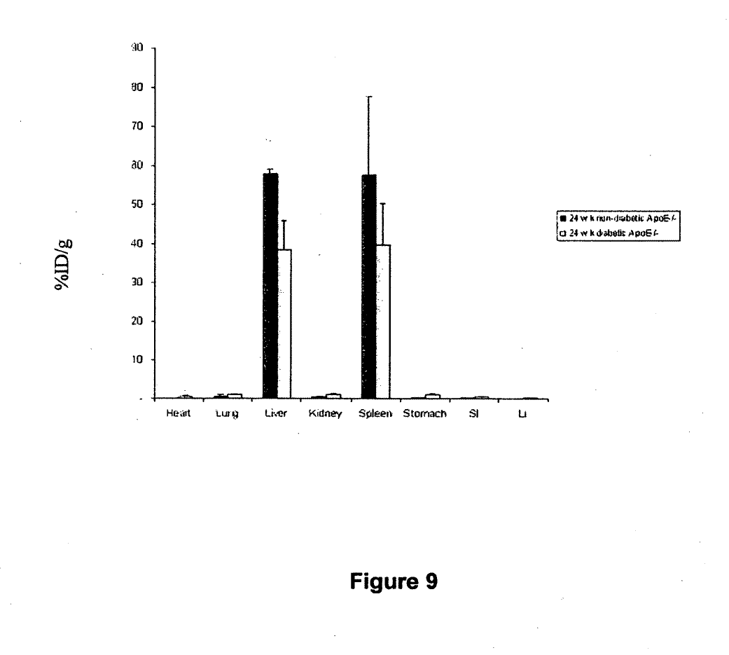

Biodistribution of radiolabeled anti-RAGE F(ab′)₂ in nontarget organs of diabetic and non-diabetic are shown in FIG. 9. The highest radiotracer uptake in both groups was in the liver and spleen.

### Histological Characterization of Atherosclerotic Lesions

Aortic sections from diabetic apoE^{-/-} mice showed mainly AHA class III lesions. Whereas in non-diabetic mice showed mainly AHA class II lesions. The mean cross sectional area of the aortic lesions, expressed as percent lesion area of total aortic area, was 37.1±16.1% (range 16.4-66.5%) in diabetic apoE^{-/-} mice and 20.7±9.5% (range 8.9-33.5) in non-diabetic apoE^{-/-} mice. (P=0.04) The control apoE^{-/-}/RAGE^{-/-} mice showed minimal or no lesions (FIG. 7A-7C).

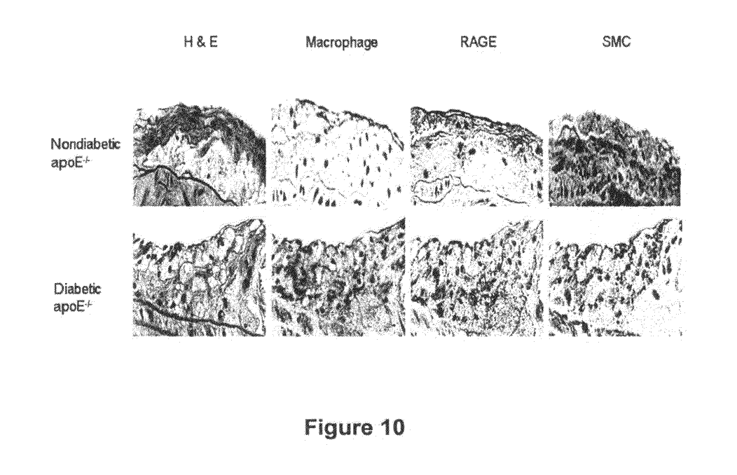

Immunohistochemical staining of the proximal aorta identified higher expressions of macrophages and RAGE in diabetic apoE^{-/-} mice compared with non-diabetic mice (FIG. 10A). The specificity of anti-RAGE antibody was confirmed by lack of staining of aortic lesions in control apoE^{-/-}/RAGE^{-/-} mice (FIG. 10B). Total RAGE and macrophage burden in the atherosclerotic lesions was quantified. The percentage of RAGE-positive cells in the lesions in diabetic apoE^{-/-} mice (37.36±6.48%) was increased approximately 2-fold compared with non-diabetic mice (18.45±3.96%) (P<0.0001). Similarly, total macrophage burden was increased in the lesions in diabetic mice (28.14±7.59%) compared with non-diabetic mice (15.25±6.29%) (P=0.006).

Quantitative ^{99m}Tc-Anti-RAGE F(ab′)₂ Uptake vs Quantitative Histomorphometry

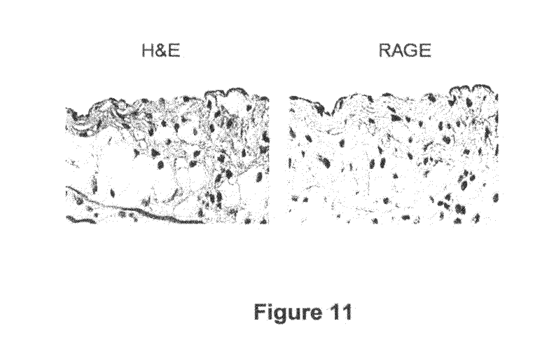

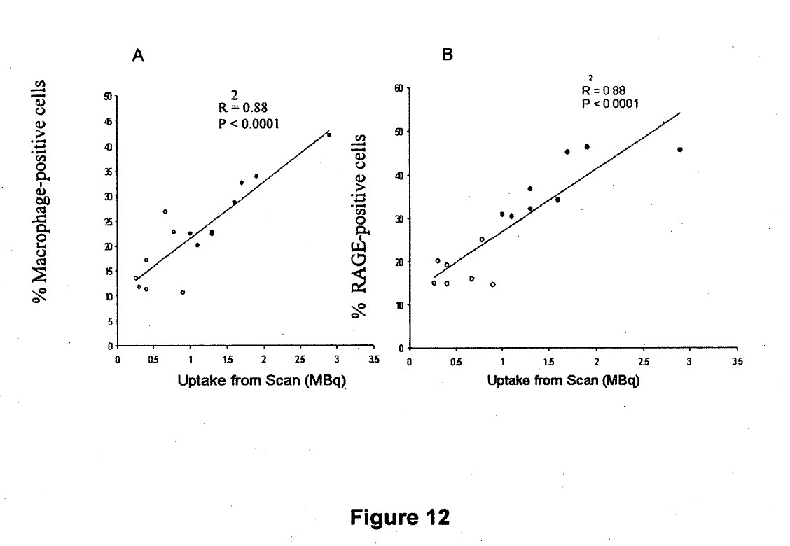

Regression analysis of values for in vivo uptake (MBq) from scans from diabetic and non-diabetic apoE^{-/-} mice demonstrated a good correlation between radiotracer uptake and RAGE expression (R²=0.88; P<0.0001) (FIG. 11A). There was also a good correlation between values for in vivo uptake from all mice and percentage of macrophages in the atherosclerotic plaques (R²=0.88; P<0.0001) (FIG. 11B).

### Discussion

This study reports for the first time the results of a study to develop a novel monoclonal antibody directed against the V-domain of RAGE designed to display immunoreactivity in mice, pigs, and humans and to radiolabel the F(ab′)₂ fragments with ^{99m}Tc and document uptake in atherosclerotic lesions of the proximal aorta in apoE^{-/-} mice with and without diabetes. The quantitative uptake of radioactivity in the target lesion correlated both with RAGE expression and with macrophages by quantitative histomorphometry.

Cardiovascular disease affects approximately 60 million people in the US. Diabetes is becoming epidemic in the US and is considered to be a “coronary artery disease equivalent” risk factor. Atherosclerosis in diabetics takes an accelerated course. Although myocardial perfusion imaging has proven prognostic usefulness there are patients with stable fixed obstructive lesions with large risk areas and stable courses and patients with <50% stenoses and no perfusion defects who have acute ischemic events including sudden death. Atherosclerosis is a widespread disease involving the entire arterial tree. Identifying plaques prone to rupture is important for event prevention. Assessing total plaque burden is important to tailor individual patient therapy. Advances in molecular biology over the past 10 years have identified potential sites in atherosclerotic plaque that can be targeted with probes that produce signals that can be detected using external imaging. Experimental and clinical studies have reported the feasibility of detecting signals from atherosclerotic plaque using nuclear medicine technology. Nuclear medicine uses probes in nanomolar concentrations that have no biological effects. Targets below the resolution of imaging devices can be detected as beacons if there are abundant binding sites and low background.

In experimental studies inflammation has been targeted with F-18 FDG, apoptosis in the plaque with annexin-V, and metalloproteinase expression with radiolabeled broad based MPI. (16-18) This study extends targeted imaging of atherosclerosis to imaging RAGE. Advanced Glycation Endproducts (AGEs) are formed by the nonenzymatic linkage of glucose to proteins and is a direct consequence of prolonged levels of hyperglycemia in diabetes. (13-15) RAGE is a member of the immunoglobulin superfamily, comprised of an extracellular region and one V-type domain followed by two C-type domains. Binding of AGEs to receptors induces multiple signaling pathways involved in plaque progression. These receptors also bind non-AGE-related pro-inflammatory markers S100/calgranulins, amphterins, and EN-RAGE. (6,7) Because of this latter broader function these receptors are implicated in progression of atherosclerosis in non-diabetics. In previous studies, a soluble form of RAGE (s-RAGE) that includes the extracellular ligand-binding domain was developed and tested and showed that administration of s-RAGE suppresses atherosclerosis in diabetic apoE^{-/-} mice and to a lesser degree in euglycemic apoE^{-/-} mice.

RAGE is highly conserved across species. Several reports using human material have studied RAGE expression in atherosclerotic plaques. (4,5) One study used plaques obtained from patients undergoing carotid endarterectomy and the other used coronary arteries from subjects who had sudden cardiac death. Both studies showed greater immunoreactivity for RAGE in atherosclerotic tissue from diabetic compared to non-diabetic patients. In addition there were greater numbers of inflammatory cells (macrophages, T lymphocytes) in the plaques and the cells stained positive for RAGE.

In a previous publication our group documented uptake of ^{99m}Tc-labeled F(ab′)₂ fragments of polyclonal anti-RAGE antibodies in the proximal aortae of 20 wk apoE^{-/-} mice fed a Western diet. Polyclonal antibodies show nonspecific uptake and are inferior to monoclonal antibodies for targeted imaging. This present report extends our previous work in several important ways. We used a newly develop monoclonal anti-RAGE antibody which shows improved specificity compared to the polyclonal antibody. We compared uptake in proximal aortic atherosclerotic lesions in apoE^{-/-} mice both with and without streptozotocin-induced diabetes. Most importantly we performed SPECT imaging and correlated the radiotracer uptake in the lesion from the scan with quantitative staining both for RAGE and for macrophages. There were excellent correlations with both variables across a range of lesion size and histology including both the diabetic and non-diabetic mice. These findings support the potential value of this radiotracer to quantify RAGE expression in atherosclerosis on in vivo nuclear imaging.

### REFERENCES

1. Kislinger T, Fu C, Huber B, Qu W, Taguchi A, Yan S D, Hofmann M, Yan S F, Pischetsrieder M, Stern D, Schmidt A M. (carboxymethyl) lysine adducts of proteins are ligands for receptor for advanced glycation endproducts that activate cell signaling pathways and modulate gene expression. J Biol Chem. 1999; 274:31740-31749.
2. Hofmann M A, Drury S, Fu C, Qu W, Taguchi A, Lu Y, Avila C, Kambham N, Bierhaus A, Nawroth P, Neurath M F, Slattery T, Beach D, McClary J, Nagashima M, Morser J, Stern D, Schmidt A M. RAGE mediates a novel proinflammatory axis: a central cell surface receptor for S100/calgranulin polypeptides. Cell. 1999; 97:889-901.
3. Schmidt A M, Yan S D, Brett J, Mora R, Nowygrod R, Stern D. Regulation of human mononuclear phagocyte migration by cell surface binding proteins for AGE. J. Clin. Invest. 1993; 91:2155-2168.
4. Burke A P, Kolodgie F D, Zieske A, Fowler D R, Weber D K, Varghese P J, Farb A, Virmani R. Morphologic findings of coronary atherosclerotic plaques in diabetes. Arterioscler Thromb Biol. 2004; 24:1266-1271.
5. Cipollone F, Iezzi A, Fazia M, Zucchelli M, Pini B, Cuccurullo C, De Cesare D, De Blasis G, Muraro R, Bei R, Chiarelli F, Schmidt A M, Cuccurullo R, Mezzetti A. The receptor RAGE as a progression factor amplifying arachidonate-dependent inflammatory and proteolytic response in human atherosclerotic plaques—Role of glycemic control. Circulation. 2003; 108:1070-1077.
6. Brett J, Schmidt A M, Yan S D, Zou Y S, Weidman E, Pinsky D, Nowygrod R, Neeper M, Przysiecki C, Shaw A, Migheli A, Stern D. Survey of the distribution of a newly characterized receptor for advanced glycation endproducts in tissues. Am J Pathol. 1993; 143:1699-1722.
7. Arumugam T, Simeone D M, Schmidt A M, Logsdon C D. S100P stimulates cell proliferation and survival via receptor for advanced glycation endproducts (RAGE). J Bio Chem. 2004; 279:5059-5065.
8. Bucciarelli L G, Wendt T, Qu W, Lu Y, Lalla E, Rong L L, Goova M T, Moser B, Kislinger T, Lee D C, Kashyap Y, Stern D M, Schmidt A M. RAGE blockade stabilizes established atherosclerosis in diabetic apolipoprotein E-null mice. Circulation. 2002; 106:2827-2835.
9. Park L, Raman K G, Lee K J, Lu Y, Ferran L J, Chow W S, Stern D, Schmidt A M. Suppression of accelerated diabetic atherosclerosis by the soluble receptor for advanced glycation endproducts. Nat. Med. 1998; 4:1025-1031.
10. Development of RAGE-directed imaging of atherosclerosis plaque in a murine model of spontaneous atherosclerosis. Circulation 2008; in press.
11. Liliensiek B, Weigand M A, Bierhaus A, Nicklas W, Kasper W, Hofer S, Plachky J, Grone H J, Kurschus F C, Schmidt A M, Yan S D, Martin E, Schleicher E, Stern D M, Hammerling G G, Nawroth P P, Arnold B. Receptor for advanced glycation endproducts (RAGE) regulates sepsis but not the adaptive immune response. J Clin Invest 2004; 113:1641-1650.
12. Candido R, Jandeleit-Dahm K A, Cao Z, Nesteroff S P, Burns W C, Twigg S M, Dilley R J, Cooper M E, Allen T J. Prevention of accelerated atherosclerosis by angiotensin-converting enzyme inhibition in diabetic apolipoprotein E-deficient mice. Circulation 2002; 106:246-253.
13. Neeper M, Schmidt A M, Brett J, Yan S D, Wang F, Pan Y C, Elliston K, Stern D, Shaw A. Cloning and expression of RAGE: a cell surface receptor for advanced glycosylation end products of proteins. J Biol Chem. 1992; 267:14998-15004.
14. Hnatowich D L, Layne W W, Childs R L, Lateinge D, Davis M A, Griffin T W, Doherty P W. Radioactive labeling of antibody: a simple and efficient method. Science. 1983; 220:613-615.
15. Daugherty A, Whitman S C. “Quantification of atherosclerosis in mice.” Methods in Molecular Biology, ed M. H. Hofker and J van Duersen. 2003; 209:293-307.
16. Tawakol A, Migrino R Q, Hoffmann U, Abbara S, Houser S, Gerwitz H, Muller J E, Brady T J, Fischman A F. Noninvasive in vivo measurement of vascular inflammation with F-18 fluorodeoxyglucose positron emission tomography. J Nucl Cardiol 2005; 12:294-301.
17. Kolodgie F D, Petrov A, Virmani R, Narula M, Verjans J; Weber D K, Hartung D, Steinmetz N, Vanderheyden J L, Vannan M, Gold H K, Reutelingsperger C P M, Hofstra Leo, Narula J. Targeting of apoptotic macrophages and experimental atheroma. Circulation. 2003; 108:3134-3139.
18. Scharfers M, Remann B, Kopha K, Breyholz H J, Wagner S, Schafers K P, Law M P, Schober O, Levkau B. Scintigraphic imaging of matrix metalloproteinase activity in the arterial wall in vivo. MMPs. Circulation. 2004; 109:2554-2559.
19. Schmidt A M, Vianna M, Gerlach M, Brett J, Ryan J, Kao J, Esposito C, Hegarty H, Hurley W, Clauss M, Wang F, Pan Y C, Tsang T C, Stern D. Isolation and characterization of binding proteins for advanced glycosylation end products from bovine lung which are present on the endothelial cell surface. Biol Chem. 1992;

1. An antibody raised to a peptide, the sequence of which is: N-R-N-G-K-E-T-K-S-N-Y-R-V-R-V-Y-Q-I-P (SEQ ID NO:1), N-R-R-G-K-E-V-K-S-N-Y-R-V-R-V-Y-Q-I-P (SEQ ID NO:2), S-R-N-G-K-E-T-K-S-N-Y-R-V-Q-V-Y-Q-I-P (SEQ ID NO:3), N-R-R-G-K-E-V-K-S-N-Y-R-V-R-V-Y-Q-I-C (SEQ ID NO:4), or Ac-N-R-R-G-K-E-V-K-S-N-Y-R-V-R-V-Y-Q-I-C-amide (SEQ ID NO:5), or said antibody linked to a therapeutic agent, or said antibody labeled with an imageable marker.

2. The antibody of claim 1 raised to a peptide, the sequence of which is Ac-N-R-R-G-K-E-V-K-S-N-Y-R-V-R-V-Y-Q-I-C-amide (SEQ ID NO:5) or said antibody linked to a therapeutic agent, or said antibody labeled with an imageable marker.

3. The antibody of claim 1, wherein the antibody is a monoclonal antibody.

4. The antibody of claim 1, wherein the antibody is labeled with an imageable marker.

5. The antibody of claim 4, wherein the imageable marker is technetium-99m.

6. The antibody of claim 4, wherein the imageable marker is rhodamine.

7. A method for determining the location of receptor for advanced glycation endproduct (RAGE) in a mammal comprising:
(a) administering to the mammal a suitable amount of an antibody raised to a peptide the sequence of which is: N-R-N-G-K-E-T-K-S-N-Y-R-V-R-V-Y-Q-I-P (SEQ ID NO:1), N-R-R-G-K-E-V-K-S-N-Y-R-V-R-V-Y-Q-I-P (SEQ ID NO:2), S-R-N-G-K-E-T-K-S-N-Y-R-V-Q-V-Y-Q-I-P (SEQ ID NO:3), N-R-R-G-K-E-V-K-S-N-Y-R-V-R-V-Y-Q-I-C (SEQ ID NO:4), or Ac-N-R-R-G-K-E-V-K-S-N-Y-R-V-R-V-Y-Q-I-C-amide (SEQ ID NO:5,) which antibody is labeled with an imageable marker; and
(b) after a period of time sufficient to permit binding of the antibody to RAGE, detecting the location of the labeled antibody in the mammal;
thereby determining the location of RAGE in the mammal.

8. The method of claim 7, wherein the antibody is raised to a peptide, the sequence of which is Ac-N-R-R-G-K-E-V-K-S-N-Y-R-V-R-V-Y-Q-I-C-amide (SEQ ID NO:5).

9. The method of claim 7, wherein the antibody is a monoclonal antibody.

10. The method of claim 7, wherein the imageable marker is technetium-99m.

11. The method of claim 7, wherein the imageable marker is rhodamine.

12. A method for treating a RAGE-related disorder in a mammal comprising administering to the mammal a therapeutically effective amount of an antibody raised to a peptide, the sequence of which is: N-R-N-G-K-E-T-K-S-N-Y-R-V-R-V-Y-Q-I-P (SEQ ID NO:1), N-R-R-G-K-E-V-K-S-N-Y-R-V-R-V-Y-Q-I-P (SEQ ID NO:2), S-R-N-G-K-E-T-K-S-N-Y-R-V-Q-V-Y-Q-I-P (SEQ ID NO:3), N-R-R-G-K-E-V-K-S-N-Y-R-V-R-V-Y-Q-I-C (SEQ ID NO:4), or Ac-N-R-R-G-K-E-V-K-S-N-Y-R-V-R-V-Y-Q-I-C-amide (SEQ ID NO:5), linked to a therapeutic agent, thereby treating a RAGE-related disorder in the mammal.

13. The method of claim 12, wherein the antibody is raised to a peptide, the sequence of which is Ac-N-R-R-G-K-E-V-K-S-N-Y-R-V-R-V-Y-Q-I-C-amide (SEQ ID NO:5).

14. The method of claim 12, wherein the antibody is a monoclonal antibody.

15. A pharmaceutical composition comprising the antibody of claim 1, wherein the antibody is linked to a therapeutic agent; and a pharmaceutically acceptable carrier.

16. The pharmaceutical composition of claim 15, wherein the antibody is raise to a peptide, the sequence of which is Ac-N-R-R-G-K-E-V-K-S-N-Y-R-V-R-V-Y-Q-I-C-amide (SEQ ID NO:5).

17. An imageable composition comprising the antibody of claim 1, wherein the antibody is labeled with an imageable marker.

18. The imageable composition of claim 17, wherein the antibody is raise to a peptide, the sequence of which is Ac-N-R-R-G-K-E-V-K-S-N-Y-R-V-R-V-Y-Q-I-C-amide (SEQ ID NO:5).

19-26. (canceled)
